(19) United States
(12) Patent Application Publication
(10) Pub. No.: US 2008/0009623 A1
(43) Pub. Date: Jan. 10, 2008

(54) Pyrimidine compounds

(75) Inventors: Yoshihisa Sano, Tsukuba-shi (JP); Eri Ena, Tsukuba-shi (JP); Yumi Asai, Tsukuba-shi (JP)

Correspondence Address: BIRCH STEWART KOLASCH & BIRCH, PO BOX 747, FALLS CHURCH, VA, 22040-0747, US

(73) Assignee: Eisai R&D Management Co., Ltd.

(21) Appl. No.: 11/808,341

(22) Filed: Jun. 8, 2007

Related U.S. Application Data
(60) Provisional application No. 60/830,352, filed on Jul. 13, 2006.

(30) Foreign Application Priority Data
Jul. 6, 2006 (JP) P2006-186980

(51) Int. Cl. C07D 239/47

(52) U.S. Cl. 544/320

(57) ABSTRACT

5-[2-Amino-4-(2-furyl)-6-hydroxypyrimidin-5-yl]-1-methylpyridin-2(1H)-one represented by the following formula is a metabolite of 5-[2-amino-4-(2-furyl)pyrimidin-5-yl]-1-methylpyridin-2(1H)-one.

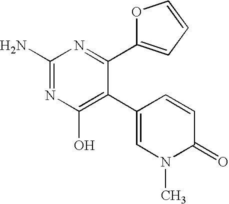

### CROSS-REFERENCE TO RELATED APPLICATIONS

This application claims priority to a U.S. provisional application No. 60/830,352 filed on Jul. 13, 2006 as well as a Japanese patent application no. 2006-186980 filed on Jul. 6, 2006, both of which are hereby incorporated by reference in their entirety.

### BACKGROUND OF THE INVENTION

1. Field of the Invention

The present invention relates to pyrimidine compounds, and more particularly, relates to 5-[2-amino-4-(2-furyl)-6-hydroxypyrimidin-5-yl]-1-methylpyridin-2(1H)-one, a salt thereof, or a hydrate of the foregoing.

2. Related Background of the Invention

5-[2-Amino-4-(2-furyl)pyrimidin-5-yl]-1-methylpyridin-2(1H)-one is a compound having adenosine receptor antagonism and being effective for prevention and treatment for various diseases such as constipation (see patent document 1).

Patent document 1: WO 03/035639

### SUMMARY OF THE INVENTION

When a certain compound is administrated to a living organism, the metabolite could show the same pharmacological effect as that of the compound which is administrated. However, patent document 1 does not disclose the metabolite. Therefore, the object of the present invention is to provide a metabolite of 5-[2-amino-4-(2-furyl)pyrimidin-5-yl]-1-methylpyridin-2(1H)-one.

As a result of much avid research, the present inventors have identified 5-[2-amino-4-(2-furyl)-6-hydroxypyrimidin-5-yl]-1-methylpyridin-2(1H)-one as a metabolite of 5-[2-amino-4-(2-furyl)pyrimidin-5-yl]-1-methylpyridin-2(1H)-one, further found that the identified compound has adenosine receptor antagonism, and have succeeded in completing this invention.

Specifically, the present invention provides 5-[2-amino-4-(2-furyl)-6-hydroxypyrimidin-5-yl]-1-methylpyridin-2(1H)-one represented by the following formula (hereinafter referred to as “the compound of the present invention”), a salt thereof, or a hydrate of the foregoing.

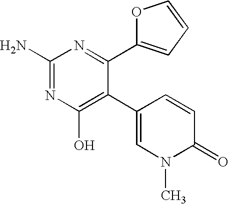

Although the compound of the present invention is formally encompassed by the claims of WO 03/035639, it is not disclosed as a specific compound.

The compound of the present invention has adenosine receptor antagonism and being effective for prevention and treatment for various diseases such as constipation.

### BRIEF DESCRIPTION OF THE DRAWINGS

FIG. 1 shows HPLC chromatogram of the rat urine sample.

### DESCRIPTION OF THE PREFERRED EMBODIMENTS

Production Method for the Compound of the Present Invention

Although the compound of the present invention is a metabolite of 5-[2-Amino-4-(2-furyl)pyrimidin-5-yl]-1-methylpyridin-2(1H)-one, the production method thereof is disclosed in Patent document 1, and it can be produced with the similar method. The entirety of the disclosure of Patent document 1 is incorporated by reference into the disclosure of the present specification.

In addition, the compound of the present invention can be produced by administrating 5-[2-Amino-4-(2-furyl)pyrimidin-5-yl]-1-methylpyridin-2(1H)-one to animals, preferably mammals, recovering excrement such as urine, and purifying by the method such as described in the following Examples. The compound of the present invention can also be produced by treating with liver microsome, and purifying, instead of administrating 5-[2-Amino-4-(2-furyl)pyrimidin-5-yl]-1-methylpyridin-2(1H)-one to animals

A “salt” as referred to throughout the present specification is not particularly restricted so long as it is formed with the compound of the invention and is pharmacologically acceptable, and as examples there may be mentioned inorganic acid salts, organic acid salts, inorganic base salts, organic base salts and acidic or basic amino acid salts.

As preferred examples of inorganic acid salts there may be mentioned hydrochloric acid salts, hydrobromic acid salts, sulfuric acid salts, nitric acid salts and phosphoric acid salts, and as preferred examples of organic acid salts there may be mentioned acetic acid salts, succinic acid salts, fumaric acid salts, maleic acid salts, tartaric acid salts, citric acid salts, lactic acid salts, stearic acid salts, benzoic acid salts, methanesulfonic acid salts and p-toluenesulfonic acid salts.

As preferred examples of inorganic base salts there may be mentioned alkali metal salts such as sodium salts and potassium salts, alkaline earth metal salts such as calcium salts and magnesium salts, and aluminum salts and ammonium salts, and as preferred examples of organic base salts there may be mentioned diethylamine salts, diethanolamine salts, meglumine salts and N,N′-dibenzylethylenediamine salts.

As preferred examples of acidic amino acid salts there may be mentioned aspartic acid salts and glutamic acid salts, and as examples of basic amino acid salts there may be mentioned arginine salts, lysine salts and ornithine salts.

When the compound of the present invention is obtained as a free form, it can be converted to a salt or a hydrate in the usual manner. In addition, when the compound of the present invention is obtained as a salt or hydrate, it can be converted to a free form in the usual manner.

Pharmaceutical Composition Comprising the Compound of the Present Invention

The use of 5-[2-amino-4-(2-furyl)-6-hydroxypyrimidin-5-yl]-1-methylpyridin-2(1H)-one as a therapeutic agent for constipation is disclosed in patent document 1, and the compound of the present invention may be used in a similar fashion as the active ingredient of a therapeutic agent for constipation.

The compound of the present invention may be formulated by an ordinary method into tablets, powder, fine powder, granules, coated tablets, capsules, syrup, lozenges, inhalation, suppository, injection, ointment, eye ointment, eye drop, nose drop, ear drop, pap, lotion or the like. For formulation there may be employed commonly used excipients, binders, lubricants, coloring agents, taste correctives and, if necessary, stabilizers, emulsifiers, absorption accelerators, surfactants, pH adjustors, antiseptics, antioxidants and the like, while other components ordinarily used as starting materials for drug formulation may also be added according to common procedures.

As examples of such components there may be mentioned animal or vegetable oils such as soybean oil, beef tallow and synthetic glycerides; hydrocarbons such as liquid paraffin, squalene and solid paraffin; ester oils such as octyldodecyl myristate and isopropyl myristate; higher alcohols such as cetostearyl alcohol and behenyl alcohol; silicone resins; silicone oils; surfactants such as polyoxyethylene fatty acid ester, sorbitan fatty acid ester, glycerin fatty acid ester, polyoxyethylenesorbitan fatty acid ester, polyoxyethylene hydrogenated castor oil and polyoxyethylene-polyoxypropylene block copolymer; water-soluble polymers such as hydroxyethylcellulose, polyacrylic acid, carboxyvinyl polymer, polyethylene glycol, polyvinylpyrrolidone and methylcellulose; lower alcohols such as ethanol and isopropyl alcohol; polyhydric alcohols such as glycerin, propylene glycol, dipropylene glycol and sorbitol; sugars such as glucose and sucrose; inorganic powders such as silicic anhydride, magnesium aluminum silicate and aluminum silicate, purified water, and the like.

As examples of excipients there may be mentioned lactose, corn starch, white soft sugar, glucose, mannitol, sorbit, crystalline cellulose, silicon dioxide and the like, as examples of binders there may be mentioned polyvinyl alcohol, polyvinyl ether, methylcellulose, ethylcellulose, gum Arabic, tragacanth gum, gelatin, shellac, hydroxypropylmethylcellulose, hydroxypropylcellulose, polyvinylpyrrolidone, polypropylene glycol-polyoxyethylene block polymer, meglumine and the like, as examples of disintegrators there may be mentioned starch, agar, gelatin powder, crystalline cellulose, calcium carbonate, sodium hydrogencarbonate, calcium citrate, dextrin, pectin, carboxymethylcellulose calcium and the like, as examples of lubricants there may be mentioned magnesium stearate, talc, polyethylene glycol, silica, hydrogenated vegetable oils and the like, as examples of coloring agents there may be mentioned those approved for addition to pharmaceuticals, and as examples of taste correctives there may be mentioned cocoa powder, menthol, aromatic powder, peppermint oil, camphor, cinnamon powder and the like.

For production of an oral preparation, the compound of the present invention may be combined with an excipient and, if necessary, a binder, disintegrator, lubricant, coloring agent, taste corrective or the like, and then made into a powder, fine powder, granules, tablets, coated tablets or capsules.

Also, there is no restriction against sugar-coating and, if necessary, other appropriate coating of the tablets or granules.

For production of a liquid preparation such as a syrup or pharmaceutical preparation for injection, the compound of the present invention may be combined with a pH adjustor, solubilizer, isotonizing agent or the like, and if necessary, with a dissolving aid, stabilizers or the like, and formulated by an ordinary method.

The method of producing an external preparation is not particularly restricted, and may be according to an ordinary method. Specifically, as base materials for pharmaceutical preparation there may be used various materials ordinarily employed for pharmaceuticals, quasi drugs, cosmetics and the like. As examples of specific base materials to be used there may be mentioned materials such as animal and vegetable oils, mineral oils, ester oils, waxes, higher alcohols, fatty acids, silicone oils, surfactants, phospholipids, alcohols, polyhydric alcohols, water-soluble polymers, clay minerals, purified water and the like, and if necessary there may also be added pH adjustors, antioxidants, chelating agents, antiseptic/mildew resistant agents, coloring agents, aromatics and the like, although base materials for an external preparation of the invention are not limited to these. If necessary there may also be included components such as circulation promoters, bactericidal agents, antiflash agents, cell activators, vitamins, amino acids, humectants, keratolytic agents and the like. The amounts of such base materials are the amounts which give concentrations indicated for production of ordinary external preparations.

The form of administration of the compound of the present invention is not particularly restricted, and may be oral administration or parenteral administration by an ordinarily employed method. For example, the crystals may be administered after formulation into tablets, powder, granules, capsules, syrup, lozenges, inhalation, suppository, injection, ointment, eye ointment, eye drop, nose drop, ear drop, pap, lotion or the like. The dosage of a pharmaceutical according to the invention may be appropriately selected depending on patient age, gender, body weight, severity of symptoms, particular type of condition, and on the type of dosage form or salt.

For example, it will generally be administered once or divided over several times at about 30 μg to 10 g, preferably 100 μg to 5 g, more preferably 100 μg to 100 mg per day for adults in the case of oral administration, or be administered once or divided over several times at about 30 μg to 1 g, preferably 100 μg to 500 mg, more preferably 100 μg to 30 mg per day for adults in the case of injection.

### EXAMPLES

[Preparation of a Metabolite and Determination of a Structure Thereof

A suspension of 5-[2-amino-4-(2-furyl)pyrimidin-5-yl]-1-methylpyridin-2(1H)-one in 5% aqueous methylcellulose solution is administrated to a male Crj:CD(SD)IGS rat (Charles River Laboratories Japan, Inc.) at 3 mg/kg body weight. The rat urine was collected for 48 hours after administration, and the rat urine sample was prepared by adding purified water (about 1 mL) to the rat urine (about 1 mL) which was collected in 0 to 8 hours after administration. This rat urine sample was analyzed by LC-MS and the metabolite having a peak at m/e (ESI)=285 (MH+) was detected.

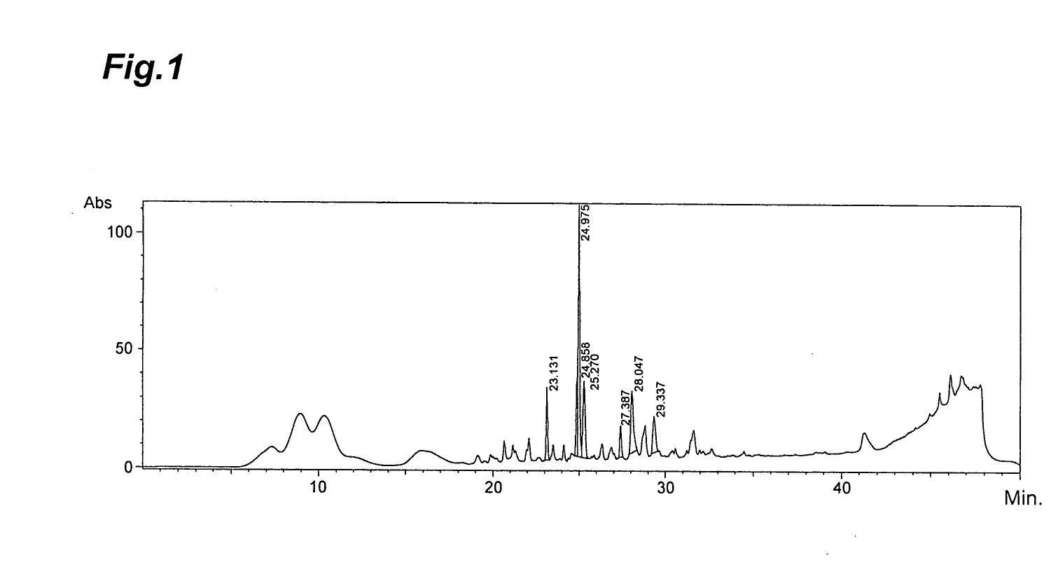

Further, the rat urine sample was analyzed by HPLC with the following conditions. As a result, the metabolite which was detected by LC-MS analysis was found at 23.1 minutes and was collected. The HPLC chromatogram is shown in FIG. 1. The obtained metabolite was analyzed by NMR, and it is identified as 5-[2-amino-4-(2-furyl)-6-hydroxypyrimidin-5-yl]-1-methylpyridin-2(1H)-one.

¹H NMR (500 MHz, DMSO-d₆) δ ppm; 7.66 (1H, broad s), 7.51 (1H, d, J=3 Hz), 7.11 (1H, dd, J=9 Hz, 3 Hz), 6.64 (2H, broad s), 6.59 (1H, d, J=3 Hz), 6.51 (1H, m), 6.33 (1H, d, J=9 Hz), 3.41 (3H, s).

[HPLC Conditions]
Apparatus: Co-sense system (HPLC system) (from Shimadzu Corporation)
Column: YMC-Pack pro C18 250×4.6 mm I.D. S-5 μm
Column temperature: 30° C. Detector: PDA (wavelength: 254 nm)
Flow rate: 1 mL/min
Injection volume: 20 μL
   Mobile phase:
Solvent A: H₂O/CH₃CN/HCOOH=990/10/1 (v/v/v)
Solvent B: CH₃CN/H₂O/HCOOH=990/10/1 (v/v/v)

### Gradient:

| Time (min.) | Concentration of Solvent B (%) |
|---|---|
| 0 | 0 |
| 3 | 0 |
| 30 | 50 |
| 30.01 | 100 |
| 35 | 100 |
| 35.01 | 0 |
| 45 | 0 |
| 45.01 | stop |

### Test Example 1

### Adenosine A₁ Receptor Binding Experiment

Human adenosine A₁ receptor cDNA was subjected to an over-expression in CHOK1 cells and its membrane specimen was suspended in a 20 mM HEPES buffer (containing 10 mM MgCl₂ and 100 mM NaCl; pH 7.4) to make the protein concentration of 66.7 μg/mL. To 0.45 mL of this membrane specimen suspension were added 0.025 mL of tritium-labeled 60 nM chlorocyclopentyl adenosine (³H-CCPA, manufactured by NEN corporation) and 0.025 mL of a solution of the compound of the present invention. The mixture was allowed to stand at 30° C. for 120 minutes, subjected to a quick suction on a glass fiber filter (GF/B; manufactured by Whatman) and immediately washed with 5 mL of water-cooled 50 mM Tris-HCl buffer twice. After that, the glass fiber filter was transferred to a vial bottle, a scintillator was added, and the radioactivity on the filter was measured by a liquid scintillation counter. Calculation of the inhibition rate of the compound of the present invention to the receptor bond of A₁ of ³H-CCPA was carried out by the following expression, and based upon that, 50% inhibitory concentration (IC₅₀) was calculated.

Inhibition Rate (%)=[1−{(Binding amount in the presence of the compound of the present invention−Non-specific binding amount)/(Total binding amount−Non-specific binding amount)}×100

The inhibition constant (Ki value) was calculated from Cheng-Prusoff's expression, and the calculated value was 157.2 nM. At this point, total binding amount is a ³H-CCPA binding radioactivity in the absence of the compound of the present invention, non-specific binding amount is a ³H -CCPA binding radioactivity in the presence of 100 μM of RPIA ([R]-[1-methyl-2-phenylethyl]adenosine), and binding amount in the presence of the compound of the present invention is a ³H-CCPA binding radioactivity in the presence of the compound of the present invention of various concentrations.

### Test Example 2

### Inhibition Experiment of A_{2B} Receptor Expressed Cells for NECA-Stimulated cAMP Production)

Human adenosine A_{2B} receptor was over-expressed in CHOK1 cells. The cells were uniformly placed on a 24-well plate at the rate of 1.5×10⁵ cells/well, incubated for one night and then used for the experiments. The inhibition rate of the compound of the present invention for the amount of cAMP produced by stimulation of 30 nM of 5′-N-ethylcarboxyamideadenosine (NECA; manufactured by Sigma corporation) was evaluated as affinity of the compound of the present invention to the A_{2B} receptor. Thus, after washing adherent cells with 2 mL/well of a Krebs-Ringer buffer (containing 0.1 % BSA; pH7.4) twice, a pre-incubation was carried out at 0.5 mL/well for 30 minutes. After that, 0.1 μL/well of a mixed solution containing NECA and the compound of the present invention were added in the presence of Ro-20-1724 (manufactured by RBI corporation) which is a phosphodiesterase inhibitor. 15 minutes after pre-incubation, the reaction was stopped by adding 0.1N HCl to the reaction solution. Measurement of cAMP was carried out using a cAMP enzyme immunoassay kit manufactured by Amersham corporation. Calculation of the inhibition rate of the compound of the present invention to the NECA-stimulated cAMP production was carried out by the following expression. Based upon that, 50% inhibitory concentration (IC₅₀) was calculated, and the calculated value was 72.7 nM.

Inhibition Rate (%)=[1−{(cAMP amount in the presence of NECA and the compound of the present invention−cAMP amount in the case of Krebs-Ringer buffer only)/(cAMP amount stimulated only by NECA−cAMP amount in the case of Krebs-Ringer buffer only)}×100

What is claimed is:

1. 5-[2-Amino-4-(2-furyl)-6-hydroxypyrimidin-5-yl]-1-methylpyridin-2(1H)-one represented by the following formula, a salt thereof, or a hydrate of the foregoing. [chemical structure]
